(19) United States
(12) Patent Application Publication
(10) Pub. No.: US 2009/0123518 A1
(43) Pub. Date: May 14, 2009

(54) Biodegradable implants with controlled bulk density

(76) Inventors: Su Il Yum, Los Altos, CA (US); Sanjay Goskonda, Sunnyvale, CA (US); Huey-Ching Su, San Jose, CA (US)

Correspondence Address: DURECT CORPORATION;THOMAS P. MCCRACKEN, 2 RESULTS WAY, CUPERTINO, CA, 95014, US

(21) Appl. No.: 12/288,537

(22) Filed: Oct. 20, 2008

Related U.S. Application Data
(60) Provisional application No. 60/999,609, filed on Oct. 18, 2007.

(51) Int. Cl. A61F 2/00; A61K 38/22

(52) U.S. Cl. 424/423; 514/12

(57) ABSTRACT

Disclosed solid water permeable implants that include a water permeable polymer and an osmotically active drug formulation that comprises a drug; wherein the solid water permeable implant has a ratio R of bulk density of the solid water permeable implant to osmotic pressure of the drug formulation wherein R is greater than about 0.244 grams/milliliter-atm. Also disclosed are methods of making and using such solid water permeable implants.

### FIELD OF THE INVENTION

In an aspect, the invention relates to solid water permeable; particularly solid water permeable implants that include a water permeable polymer and an osmotically active drug formulation that comprises a drug.

### BACKGROUND OF THE INVENTION

Continuous, long term drug delivery methodologies may have certain advantages in that they may achieve a desired blood level of the drug in circulation for an extended period of time. A number of modes of administration of continuous dose, long-term delivery devices have been used or proposed. One of these is the use of subcutaneous implants, which offers a particularly desirable combination of properties to permit the administration of substances on a localized or systemic basis. To this end, subcutaneous implants serving as depots capable of slow release of a drug have been proposed. These implants suggest the possibility of attaining continuous administration over a prolonged period of time to achieve a relatively uniform delivery rate and, if desired, a static blood level. Since an excessive concentration of drug never enters the body fluids, problems of pulse entry are overcome and metabolic half-life is not a factor of controlling importance.

Despite the advantages of administering drugs from implants, prior art devices designed for this purpose have possessed one or more disadvantages which limit their acceptability and efficacy. Among such disadvantages are nonbiodegradability which may require a surgical procedure to remove them; nonbiocompatibility which may result in the introduction of undesirable and even harmful substances into the body; antigenicity which gives rise to the production of unwanted antigen bodies in the system; and difficulty in controlling release rates of the drugs. Additionally, conventional delivery devices may not provide sufficient long-term drug delivery rates to facilitate long-term dosing, and may suffer from undesirably high one day cumulative drug release. Such a high one day cumulative drug release can produce adverse events in subject to which the conventional device is administered due to high systemic drug levels.

What is needed are compositions and methods that address the problems noted above.

### SUMMARY OF THE INVENTION

In an aspect, the invention relates to a method comprising: providing a solid water permeable implant comprising a water permeable polymer and an osmotically active drug formulation that comprises a drug; wherein the solid water permeable implant has a ratio R of bulk density of the solid water permeable implant to osmotic pressure of the drug formulation wherein R is greater than about 0.244 grams/milliliter-atm; administering the solid water permeable implant to a subject; and sustainably releasing the drug from the solid water permeable implant for at least about one week following administration of the solid water permeable implant.

In another aspect, the invention relates to a method comprising: forming a solid water permeable implant comprising a water permeable polymer and an osmotically active drug formulation that comprises a drug; wherein the solid water permeable implant has a ratio R of bulk density of the solid water permeable implant to osmotic pressure of the drug formulation wherein R is greater than about 0.244 grams/milliliter-atm.

### DETAILED DESCRIPTION FO THE INVENTION

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting.

All publications, patents and patent applications cited herein, whether supra or infra, are hereby incorporated by reference in their entirety for all purposes.

As used in this specification and the appended claims, the singular forms “a,” “an” and “the” include plural referents unless the content clearly dictates otherwise. For example, reference to “a polymer” includes a mixture of two or more such molecules, reference to “a solvent” includes a mixture of two or more such compositions, reference to “an adhesive” includes mixtures of two or more such materials, and the like.

### A. INTRODUCTION

The inventors have unexpectedly discovered that the aforementioned problems in the art may be addressed by providing methods that comprise providing a solid water permeable implant comprising a water permeable polymer and an osmotically active drug formulation that comprises a drug; wherein the solid water permeable implant has a ratio R of bulk density of the solid water permeable implant to osmotic pressure of the drug formulation wherein R is greater than about 0.244 grams/milliliter-atm; administering the solid water permeable implant to a subject; and sustainably releasing the drug from the solid water permeable implant for at least about one week following administration of the solid water permeable implant. Further inventors have unexpectedly discovered that the aforementioned problems in the art may be addressed by providing methods that comprise forming a solid water permeable implant comprising a water permeable polymer and an osmotically active drug formulation that comprises a drug; wherein the solid water permeable implant has a ratio R of bulk density of the solid water permeable implant to osmotic pressure of the drug formulation wherein R is greater than about 0.244 grams/milliliter-atm.

The inventors have determined that the bulk density of solid water permeable implants can be an important factor in determining drug release performance, particularly one day cumulative drug release performance, of the implants. In particular, for implants that comprise an osmotically active drug formulation, the ratio R of bulk density of the solid water permeable implant to osmotic pressure of the drug formulation can predict drug release performance of the implants.

As an example of this discovery, the inventors selected leuprolide acetate as a sample compound. The inventors then experimentally determined that the osmotic pressure of leuprolide acetate in water is approximately 5 atmospheres at room temperature.

Next, in Examples 1-5 (Trials 1-17, with the individual Trial data being reported in Table 1), the inventors determined that when the bulk density of solid water permeable implants is less than 1.22 grams/milliliter, the one day cumulative drug release averaged 5.77 weight percent, based on the initial total weight of drug present in the solid water permeable implant. In contrast, the inventors determined that when the bulk density of solid water permeable implants is greater than 1.22 grams/milliliter, the one day cumulative drug release averaged only 2.73 weight percent, based on the initial total weight of drug present in the solid water permeable implant. In other words, the average one day cumulative drug release is approximately two times greater for solid water permeable implants having a bulk density less than 1.22 grams/milliliter than for solid water permeable implants having a bulk density greater than 1.22 grams/milliliter. This bulk density cut off point may then be ratioed against the osmotic pressure of the drug in question to arrive at a unitless quantity that can be used to characterize a solid water permeable implant with superior performance properties. Methods and materials for making and using such solid water permeable implants are further described herein.

The invention will now be described in more detail.

### B. DEFINITIONS

All percentages are weight percent unless otherwise noted.

All references cited herein are incorporated herein by reference in their entirety and for all purposes to the same extent as if each individual publication or patent or patent application was specifically and individually indicated to be incorporated by reference in its entirety for all purposes and/or reproduced fully herein. The discussion of references herein is intended merely to summarize the assertions made by their authors and no admission is made that any reference constitutes prior art. Applicants reserve the right to challenge the accuracy and pertinence of the cited references.

The present invention is best understood by reference to the following definitions, the drawings and exemplary disclosure provided herein.

“Solid” means that an object or material has a definite shape and volume; such an object or material is neither liquid or gaseous.

“Water permeable” means that an object or material possesses the property of allowing water to penetrate or pass through the object or material.

“Implant” means a mass placed or formed inside a subject for the purpose of sustainably releasing a drug from the implant.

“Biodegradable” means a material such as a polymer that will degrade or erode in vivo to form smaller chemical species, wherein the degradation can result, for example, from enzymatic, chemical, and physical processes.

“Biocompatible” means a material such as a polymer and any degradation products of the material that are non toxic to a subject and present no significant, deleterious or untoward effects on the subject's body.

“Polymer” means a naturally occurring or synthetic compound made up of a linked series of repeat units. Polymer(s) include, but are not limited to, thermoplastic polymers and thermoset polymers. Polymer(s) may comprise linear polymers and/or branched polymers. Polymers may be synthesized from a single species of monomers, or may be copolymers that may be synthesized from more than one species of monomers. In certain preferred embodiments, the polymer may be biocompatible and/or biodegradable.

Examples of suitable polymers, preferably biocompatible and/or biodegradable polymers include but are not limited to polyhydroxy acids, such as poly(lactide)s, poly(glycolide)s, poly(lactide-co-glycolide)s, poly(lactic acid)s, poly(glycolic acid)s, and poly(lactic acid-co-glycolic acid)s, polyanhydrides, polyorthoesters, polyetheresters, polyethylene glycol, polycaprolactone, polyesteramides, polyphosphazines, polycarbonates, polyamides, and copolymers and blends thereof. Preferred materials are polycaprolactone, poly(lactide)s, poly(glycolide)s, and copolymers thereof. Representative natural polymer materials include polysaccharides and proteins.

“Osmotically active” means a material that generates an osmotic pressure across a semipermeable membrane.

“Drug formulation” means a pharmaceutical composition that comprises a drug, and that is useful in the practice of this invention.

“Drug” means any substance used internally or externally as a medicine for the treatment, cure, or prevention of a disease or disorder, and includes but is not limited to immunosuppressants, antioxidants, anesthetics, chemotherapeutic agents, steroids (including retinoids), hormones, antibiotics, antivirals, antifungals, antiproliferatives, antihistamines, anticoagulants, antiphotoaging agents, melanotropic peptides, nonsteroidal and steroidal anti-inflammatory compounds, antipsychotics, and radiation absorbers, including UV-absorbers.

Representative therapeutic active agents include immunosuppressants, antioxidants, anesthetics, chemotherapeutic agents, steroids (including retinoids), hormones, antibiotics, antivirals, antifungals, antiproliferatives, antihistamines, anticoagulants, antiphotoaging agents, melanotropic peptides, nonsteroidal and steroidal anti-inflammatory compounds, antipsychotics, and radiation absorbers, including UV-absorbers. Other non-limiting examples of active agents include anti-infectives such as nitrofurazone, sodium propionate, antibiotics, including penicillin, tetracycline, oxytetracycline, chlorotetracycline, bacitracin, nystatin, streptomycin, neomycin, polymyxin, gramicidin, chloramphenicol, erythromycin, and azithromycin; sulfonamides, including sulfacetamide, sulfamethizole, sulfamethazine, sulfadiazine, sulfamerazine, and sulfisoxazole, and anti-virals including idoxuridine; antiallergenics such as antazoline, methapyritene, chlorpheniramine, pyrilamine prophenpyridamine, hydrocortisone, cortisone, hydrocortisone acetate, dexamethasone, dexamethasone 21-phosphate, fluocinolone, triamcinolone, medrysone, prednisolone, prednisolone 21-sodium succinate, and prednisolone acetate; desensitizing agents such as ragweed pollen antigens, hay fever pollen antigens, dust antigen and milk antigen; decongestants such as phenylephrine, naphazoline, and tetrahydrazoline; miotics and anticholinesterases such as pilocarpine, esperine salicylate, carbachol, diisopropyl fluorophosphate, phospholine iodide, and demecarium bromide; parasympatholytics such as atropine sulfate, cyclopentolate, homatropine, scopolamine, tropicamide, eucatropine, and hydroxyamphetamine; sympathomimetics such as epinephrine; sedatives and hypnotics such as pentobarbital sodium, phenobarbital, secobarbital sodium, codeine, (a-bromoisovaleryl) urea, carbromal; psychic energizers such as 3-(2-aminopropyl) indole acetate and 3-(2-aminobutyl) indole acetate; tranquilizers such as reserpine, chlorpromayline, and thiopropazate; androgenic steroids such as methyl-testosterone and fluorymesterone; estrogens such as estrone, 17-b-estradiol, ethinyl estradiol, and diethyl stilbestrol; progestational agents such as progesterone, megestrol, melengestrol, chlormadinone, ethisterone, norethynodrel, 19-norprogesterone, norethindrone, medroxyprogesterone and 17-b-hydroxy-progesterone; humoral agents such as the prostaglandins, for example PGE1, PGE2 and PGF2; antipyretics such as aspirin, sodium salicylate, and salicylamide; antispasmodics such as atropine, methantheline, papaverine, and methscopolamine bromide; antimalarials such as the 4-aminoquinolines, 8-aminoquinolines, chloroquine, and pyrimethamine, antihistamines such as diphenhydramine, dimenhydrinate, tripelennamine, perphenazine, and chlorphenazine; cardioactive agents such as dibenzhydroflume thiazide, flumethiazide, chlorothiazide, and aminotrate, natural and synthetic bioactive peptides and proteins, including growth factors, cell adhesion factors, cytokines, and biological response modifiers.

In one embodiment, the incorporated material is a vaccine and the substance to be delivered is an antigen. The antigen can be derived from a cell, bacteria, or virus particle, or portion thereof. As defined herein, antigen may be a protein, peptide, polysaccharide, glycoprotein, glycolipid, nucleic acid, or combination thereof, which elicits an immunogenic response in an animal, for example, a mammal, bird, or fish. The immunogenic response can be humoral or cell-mediated. In the event the material to which the immunogenic response is to be directed is poorly antigenic, it may be conjugated to a carrier, such as albumin, or to a hapten, using standard covalent binding techniques, for example, with one of the several commercially available reagent kits. Examples of preferred antigens include viral proteins such as influenza proteins, human immunodeficiency virus (HIV) proteins, and hepatitis A, B, or C proteins, and bacterial proteins, lipopolysaccharides such as gram negative bacterial cell walls and Neisseria gonorrhea proteins, and parvovirus.

“Bulk density” means the mass of an item per unit volume. It may be calculated using, for cylindral implants, the measured diameter and the length of the implant to determine volumer. The diameter and length of the implant can be measured by calibrated calipers. The bulk density of the implant is calculated by measuring the united weight of the implant determined by an analytical balance, divided by the calculated unit volume, determined as disclosed above.

“Osmotic pressure of the drug” means the pressure that must be applied to a solution to prevent the net flow of solvent molecules (such as water) through a semipermeable membrane from a solution of lower drug concentration to a solution of higher drug concentration. The osmotic pressure of the drug can be experimentally determined with use of a vapor pressure osmometer, such as the Vapro® vapor pressure osmometer.

“Administering” or “administration” means providing a drug to a subject in a manner that is pharmacologically useful.

“Subject” is used interchangeably with “individual” and means any human with which it is desired to practice the present invention. The term “subject” does not denote a particular age, and the present systems are thus suited for use with subjects of any age, such as infant, adolescent, adult and senior aged subjects In certain embodiments, a subject may comprise a patient.

“Sustainably releasing” or “sustained release means continuous releasing or continuous release of a drug or a dose of a drug over a continuous period of greater than about 12 hours, preferably, greater than about 24 hours, more preferably, greater than about 1 week, more preferably greater than about 2 weeks, more preferably still, greater than about 3 weeks, most preferably, greater than about 4 weeks.

### C. IMPLANTS

There are a variety of methods for making implants according to the invention.

Certain embodiments include, but are not limited to: wet spinning, dry spinning and melt spinning. Wet spinning involves extruding a solution of a polymer through an orifice into a nonsolvent to coagulate the polymer. In the dry-spinning process, a solution of the drug formulation and polymer is forced through an orifice and fed into a heated column that evaporates the solvent to form a filament. In melt-spinning, a thermoplastic polymer is heated above its melting point, extruded through an orifice together with the drug formulation and, and cooled to form a filament. If the implants is desired to be a coaxial implant, the drug may be extruded in the core of the coaxial implant at the same time as a rate-controlling polymer membrane (also referred to as a “sheath”). A typical coaxial spinneret consists of two concentric rings. The drug, either in pure form or dispersed within a polymeric or nonpolymeric matrix, is pumped through the inner ring, where it forms the core. The rate-controlling polymer is pumped through the outer ring to form the sheath. As both streams of material emerge from the spinneret, they solidify to form the coaxial implant. The rate at which the two materials are pumped to the coaxial spinneret determines the thickness of the sheath membrane and the size of the implant.

If the implant is formed by extrusion, the polymer and/or drug is liquified for extrusion either by melting or dissolution in a solvent. The preferred method of preparation of extruded implants is melt extrusion. The implant formulation is fed to an extrusion die. The diameter of the implant is controlled by the dimensions of the die, the extrusion conditions, the extrusion rates of the two extruder, and the take-off speed. In this way, the implant diameter and thickness can be controlled.

Implant may also be made by conventional compression processes that are used to make conventional oral tablets. In such processes, particles or granules comprising a drug formulation are compressed in a die between two punches to form a single compact form. The particles or granules prior to compression may be made using various technologies such as roller compaction/milling, spray drying, solvent granulation, or size reduction of larger particles. General formulation and processes for manufacturing such tablets is described in Pharmaceutical Dosage Forms: Tablets, Vol 1, Second Edition, Edited by H. A. Liberman, J. Schwartz, L. Lachman, CRC Press, 1989.

Alternatively, implants according to the invention may be made by injection molding. In an injection molding process, molten material comprising a drug formulation is injected at a high pressure into a mold, which is inverse of the implant/product shape. The molds are generally made of steel and are precision machined to obtain shape and size of the final implant. General use of polymer molding techniques for the purpose of controlled drug delivery is described in “Controlled Drug Delivery”, edited by J. R. Robinson and V. H. Lee (1978).

The drug formulation can be combined with the polymer in a variety of ways. If the polymer contains a liquid carrier then the drug formulation and polymer/carrier mixture can be mixed to form a slurry. Alternatively, the drug formulation and polymer can be mixed by solvent-blending, dry blending, or melt blending. More uniform mixing may be obtained by extruding the drug formulation-polymer matrix twice. In the preferred embodiment, the implant is formulated by dry blending the drug formulation and polymer, melt extruding the blend, and grinding the extrudate to form a feedstock for a second extrusion.

Although generally formed in a geometry where the cross-section is a circle, the implant can also be prepared with any other cross-sectional geometry, for example, an ellipsoid, a lobe, a square, or a triangle. The implant preferably has the shape of a rod, although it may also be generally sphere-shaped in certain preferred embodiments.

The drug loading in the implant may be in the range of about 0.1 to about 80 wt %, based on total weight of the implant, when either liquid carriers or polymers are used in the implant. A more preferred loading is in the range of about 10 to about 60 wt % and the most preferred loading is in the range of about 20 to about 50 wt %, based on total weight of the implant.

The implants may be prepared in a variety of sizes depending on the total dose of drug and the envisioned method of administration. In a preferred embodiment, the overall diameter is between 0.05 and 5.0 mm. For subcutaneous administration in humans, an overall diameter of between 1.0 and 4.0 mm may be more preferred. The length of the implant is typically between about 0.3 cm and 10 cm. For subcutaneous implantation, a more preferred length is between about 0.3 cm and 3.0 cm.

If the polymer and drug formulation are solvent blended, the selection of the solvent used in the process generally depends on the polymer and drug formulation chosen, as well as the particular means of solvent removal to be employed. Organic solvents, such as acetone, methyl ethyl ketone, tetrahydrofuran, ethyl lactate, ethyl acetate, dichloromethane, and ethyl acetate/alcohol blends, are preferred solvents.

Examples of suitable therapeutic and/or prophylactic active agents include proteins, such as hormones, antigens, and growth factors; nucleic acids, such as antisense molecules; and smaller molecules, such as antibiotics, steroids, decongestants, neuroactive agents, anesthetics, sedatives, and antibodies, such as antibodies that bind to growth hormone receptors, including humanized antibodies, adjuvants, and combinations thereof. Examples of suitable diagnostic and/or therapeutic active agents include radioactive isotopes and radioopaque agents.

The amount of drug to be incorporated and the amount used in the manufacturing process will vary depending upon the particular drug, the desired effect of the drug at the planned release levels, and the time span over which the drug should be released. The inventive methods can be used to incorporate more than one drug into the inventive implants. The drug also can be mixed with one or more excipients, such as stabilizing agents, known in the art.

The inventive implants may be implanted using minimally invasive procedures at a site where release is desired. These can be implanted using trocars or catheters subcutaneously, intraperitoneally, intramuscularly, and intralumenally (intravaginally, intrauterine, rectal, periodontal). The implants can be fabricated as part of a matrix, graft, prosthetic or coating, for example, intravascularly.

Additional general information regarding making of implants may be found in, for example, Cowsar and Dunn, Chapter 12 “Biodegradable and Nonbiodegradable Delivery Systems” pp. 145-162; Gibson, et al., Chapter 31 “Development of a Fibrous IUD Delivery System for Estradiol/Progesterone” pp. 215-226; Dunn, et al., “Fibrous Polymers for the Delivery of Contraceptive Steroids to the Female Reproductive Tract” pp. 125-146; Dunn, et al., “Fibrous Delivery Systems for Antimicrobial Agents” from Polymeric Materials in Medication ed. C.G. Gebelein and Carraher (Plenum Publishing Corporation, 1985) pp 47-59, U.S. Pat. Nos. 3,518,340; 3,773,919; 4,351,337; and 5,366,734; published applications WO/2004/110400 and WO/2006/071208, and published US patent application 20030007992.

### D. CONTROL OF BULK DENSITY

The inventors have identified a number of methods to control bulk density of the inventive implants. One method is to control the amount of gas in the extruder melt. This can be done in at least two ways: conditioning the feed and removing the extruder melt off-gas.

Extrusion feed materials can be conditioned by vacuum drying. In such embodiments, the feed material prior may be dried under vacuum (˜29 inches of mercury) for a minimum of 10 hours, preferably a minimum of 15 hours, and more preferably a minimum of 24 hours prior to final extrusion. In preferable embodiments, the vacuum drying may be performed at room temperature. In a more preferable embodiment, the vacuum drying may be performed at room temperature in a contained environment, such as the ante-chamber of a glovebox.

In certain embodiments, it may be desirable to remove off-gas from the extrusion melt during operation of the extruder. In such case, the extruder melt off-gas can be removed by either venting or placing under vacuum the extruder at the feed hopper or at various points along the extruder barrel.

While there has been described and pointed out features and advantages of the invention, as applied to present embodiments, those skilled in the medical art will appreciate that various modifications, changes, additions, and omissions in the method described in the specification can be made without departing from the spirit of the invention.

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods within the scope of the invention, in addition to those enumerated herein, will be apparent to those skilled in the art from the foregoing description. Such modifications and variations are intended to fall within the scope of the appended claims. The present invention is to be limited only by the terms of the appended claims, along with the full scope of equivalents to which such claims are entitled.

The following Examples are meant to be illustrative of the claimed invention, and not limiting in any way.

### E. EXAMPLES

### Example 1 (Trials 1-3)

Implants were made as follows: 25.28 grams of milled leuprolide acetate and 74.84 grams of 90/10 poly (DL-Lactide-CO-Glycolide)-Methoxypoly (ethylene-glycol) 750 were blended in a stainless steel container on a Inversina Mixer for 10 minutes.

This blend was processed through a Randcastle ⅜″ extruder with following process conditions: screw speed 10 rpm, extruder temperatures were Zone 1=175° F., Zone 2=215° F., Zone 3=238° F. and a die temperature of 238° F. This extrudate was pelletized and reduced further in size using cryogrinding with liquid nitrogen in a Retsch mill at 14000 rpm. The cryoground material was allowed to warm up under dry environment of glovebox under compressed dry air for about 18 hours in a glove box. This feed material was used for production of final implants using extrusion process.

The conditioned feedstock was introduced into a Rancastle ⅜″ single screw extruder run at 10 rpm to produce bulk rods that were cut into implants. The extruder temperatures were Zone 1=175° F., Zone 2=215° F., Zone 3=238° F., and a die temperature of 238° F. The diameter of the die was 0.059″ and final diameter of the filament was controlled to around 1.5 mm. Based on the potency of the implants the implants were cut to a length of about 11.3 mg per implant, where length was expressed as weight per implant.

Next, the bulk density of the implants was determined by using the formula ρ=m/V. where “m” is the weight of the implant in mg and “V” is the volume of the implant in mm³. The volume of the implant was calculated by using measurements of diameter and length of the implant determined using calipers.

The cumulative amount of leuprolide acetate released one day after release testing began was determined as follows. Each implant was placed into a clean scintillation vial. Then 10 mL of 67 mM phosphate buffer with 0.5% sodium azide (pH 7.4) was added to the scintillation vial. The samples were stored in a 37° C. incubator. After one day, the buffer medium was tested for the amount of leuprolide acetate released.

The bulk density and cumulative one day release data for the implant according to this Example are presented in Table 1.

### Example 2 (Trial 3)

Implants were made as follows. 24.657 grams of milled leuprolide acetate, and 70.339 grams of 90/10 poly (DL-Lactide-CO-Glycolide)-Methoxypoly(ethylene-glycol) 750 were blended in a stainless steel container on an Inversina mixer, for 10 minutes.

This blend was processed through a Randcastle ⅜″ extruder with following process conditions: screw speed 10 rpm, extruder temperatures were Zone 1=170° F., Zone 2=205° F., Zone 3=213° F. and a die temperature of 213° F. This extrudate was pelletized and reduced further in size using cryogrinding with liquid nitrogen in a Retsch mill at 8000 rpm. The cryoground material was allowed to warm up in the dry environment of a glovebox antechamber under compressed dry air for about 15 hours. This feed material was used for production of final implants using extrusion process

The feedstock was introduced into a Rancastle ⅜″ single screw extruder run at 10 rpm to produce bulk rods that were cut into implants. The extruder temperatures were Zone 1=170° F., Zone 2=205° F., Zone 3=215° F. and a die temperature of 215° F.

Next, the bulk density of the implants was determined according to the methods of Example 1. The cumulative amount of leuprolide acetate released one day after release testing began was determined according to the methods of Example 1. The bulk density and cumulative one day release data for the implant according to this Example are presented in Table 1.

### Example 3 (Trial 4)

Implants were made as follows. 39.872 grams of milled leuprolide acetate, and 110.148 grams of 90/10 poly (DL-Lactide-CO-Glycolide)-Methoxypoly (ethylene-glycol) 750 were blended in a stainless steel container in an Inversina mixer, for 10 minutes.

This blend was processed through a Randcastle ⅜″ extruder with following process conditions: screw speed 10 rpm, extruder temperatures were Zone 1=175° F., Zone 2=215° F., Zone 3=238° F. and a die temperature of 238° F. This extrudate was pelletized and reduced further in size using cryogrinding with liquid nitrogen in a Retsch mill at 14000 rpm. The cryoground material was allowed to warm up in the dry environment of a glovebox antechamber under compressed dry air for about 18 hours. This feed material was used for production of final implants using extrusion process

The feedstock was introduced into a Rancastle ⅜″ single screw extruder run at 10 rpm to produce bulk rods that were cut into implants. The original process temperatures were: Zone 1=175° F., Zone 2=215° F., Zone 3=238° F. and a die temperature of 238° F. The extrudate output was noted as being faster, and the filaments had low melt strength. In order to control the process the extruder temperatures were changed to Zone 1=167° F., Zone 2=204° F., Zone 3=227° F., and a die temperature of 227° F.

Next, the bulk density of the implants was determined according to the methods of Example 1. The cumulative amount of leuprolide acetate released one day after release testing began was determined according to the methods of Example 1. The bulk density and cumulative one day release data for the implant according to this Example are presented in Table 1.

### Example 4 (Trials 5-9)

Implants were made as follows. 36.432 grams of milled leuprolide acetate, and 113.636 grams of 90/10 poly (DL-Lactide-CO-Glycolide)-Methoxypoly (ethylene-glycol) 750 were blended in a stainless steel container in an Inversina mixer, for 10 minutes.

This blend was processed through a Randcastle ⅜″ extruder with following process conditions: screw speed 10 rpm, extruder temperatures were Zone 1=175° F., Zone 2=215° F., Zone 3=238° F. and a die temperature of 238° F. This extrudate was pelletized and reduced further in size using cryogrinding with liquid nitrogen in a Retsch mill at 14000 rpm. The cryoground material was allowed to warm up in the dry environment of a glovebox antechamber under compressed dry air for about 15 hours. This feed material was used for production of final implants using extrusion process

The feedstock was introduced into a Rancastle ⅜″ single screw extruder run at 10 rpm to produce bulk rods that were cut into implants. The extruder temperatures were Zone 1=175° F., Zone 2=215° F., Zone 3=238° F. and a die temperature of 238° F.

Next, the bulk density of the implants was determined according to the methods of Example 1. The bulk density of the implant was 1.25 mg/mm³. The cumulative amount of leuprolide acetate released one day after release testing began was determined according to the methods of Example 1. The bulk density and cumulative one day release data for the implant according to this Example are presented in Table 1.

### Example 5 (Trials 10-17)

mPEG 750 initiated 90:10 poly(DL-lactide-co-glycolide), mPEG-750 90:10 DL-PLG, having an inherent viscosity of 0.87 dL/g (CHCl3 at 30° C.) was cryogenically ground using a Retsch ZM 100 Ultracentrifugal Mill equipped with a 1-mm screen and operated at approximately 14,000 rpm. The polymer pellets were combined with liquid nitrogen (LN2) and added to the mill at a rate sufficiently slow to prevent overheating. The milled material was collected and dried under vacuum at ambient temperature for approximately 75 hrs. Next, leuprolide acetate (LA), Genzyme Pharmaceuticals Lot M0057, was milled using a Trost Gem-T Jet Mill with N2 as the carrier gas. LA (56.3 g) was pre-ground using a glass mortar and pestle and then fed to the mill using a suction feeder. The milled LA was recovered from the mill and dried under vacuum at ambient temperature for ˜70 hrs. Next, approximately 6 g of the LA and approximately 14 g of the mPEG-750 90:10 DL-PLG were combined and mixed by hand. The blend was vacuum dried at ambient temperature for approximately 46 hrs. After drying, the blend was extruded using a Randcastle 0.375-in extruder equipped with a round hole die having an opening of ˜1.6 mm. The extruder was operated at approximately 10 rpm with the following target temperatures:
Zone 1=180° F.
Zone 2=225° F.
Zone 3=248° F.
Die=248° F.
Melt=235-240° F.

The resulting rod stock was collected, broken into small pieces, and cryogenically milled as described above to yield milled material. The milled material was dried under vacuum at ambient temperature for approximately 21 hrs.

The milled LA/polymer blend was extruded a second time using the same equipment. The extruder was operated at the following target temperatures:
Zone 1=200° F.
Zone 2=225° F.
Zone 3=248° F.
Die=248° F.
Melt=251-252° F.

The screw speed was set to approximately 10 RPM initially but later slowed to 7.6 rpm to compensate for increased pressures and motor load. Steady state pressures were maintained in the range of 1600-1830 psig. The rod stock was collected in lengths of approximately 20 30 cm and stored over desiccant pending testing.

Next, the bulk density of the implants was determined according to the methods of Example 1. The cumulative amount of leuprolide acetate released one day after release testing began was determined according to the methods of Example 1. The bulk density and cumulative one day release data for the implant according to this Example are presented in Table 1.

TABLE 1
Implant Test Data

| | | | Cumulative One |
|---|---|---|---|
| Trial Number | | Bulk Density (g/cc) | Day Release (mg) |
| 1 | (Ex. 1) | 1.22 | 5.4 |
| 2 | (Ex. 1) | 1.22 | 3.9 |
| 3 | (Ex. 1) | 1.20 | 4.1 |
| 3 | (Ex. 2) | 1.21 | 7.6 |
| 4 | (Ex. 3) | 1.15 | 14.7 |
| 5 | (Ex. 4) | 1.23 | 2.2 |
| 6 | (Ex. 4) | 1.22 | 1.2 |
| 7 | (Ex. 4) | 1.24 | 1.2 |
| 8 | (Ex. 4) | 1.23 | 1.2 |
| 9 | (Ex. 4) | 1.21 | 1.2 |
| 10 | (Ex. 5) | 1.19 | 1.7 |
| 11 | (Ex. 5) | 1.21 | 20.2 |
| 12 | (Ex. 5) | 1.21 | 8.2 |
| 13 | (Ex. 5) | 1.237 | 2.6 |
| 14 | (Ex. 5) | 1.25 | 7.61 |
| 15 | (Ex. 5) | 1.25 | 5.58 |
| 16 | (Ex. 5) | 1.25 | 1.9 |
| 17 | (Ex. 5) | 1.27 | 2.7 |

What is claimed is:

1. A method comprising:
providing a solid water permeable implant comprising a water permeable polymer and an osmotically active drug formulation that comprises a drug; wherein the solid water permeable implant has a ratio R of bulk density of the solid water permeable implant to osmotic pressure of the drug formulation wherein R is greater than about 0.244 grams/milliliter-atm;
administering the solid water permeable implant to a subject; and
sustainably releasing the drug from the solid water permeable implant for at least about one week following administration of the solid water permeable implant.

2. The method of claim 1, wherein the solid water permeable implant exhibits a reduced one day cumulative drug release compared to a second solid water permeable implant comprising the water permeable polymer and the osmotically active drug formulation that comprises the drug; wherein the second solid water permeable implant has a ratio R of bulk density of the solid water permeable implant to osmotic pressure of the drug formulation wherein R is less than 0.244 grams/milliliter-atm.

3. The method of claim 1, wherein the drug is sustainably released from the solid water permeable implant for at least about two weeks following administration of the solid water permeable implant.

4. The method of claim 1, wherein the water permeable polymer comprises a poly(lactide), poly(glycolide), poly(lactide-co-glycolide), poly(lactic acid), poly(glycolic acid), poly(lactic acid-co-glycolic acid), polyanhydride, polyorthoester, polyetherester, polyethylene glycol, polycaprolactone, polyesteramide, polyphosphazine, polycarbonate, polyamide, or a copolymer or blend thereof.

5. The method of claim 1, wherein the drug comprises leuprolide acetate.

6. A method comprising:
forming a solid water permeable implant comprising a water permeable polymer and an osmotically active drug formulation that comprises a drug; wherein the solid water permeable implant has a ratio R of bulk density of the solid water permeable implant to osmotic pressure of the drug formulation wherein R is greater than about 0.244 grams/milliliter-atm.

7. The method of claim 6, further comprising administering the formed solid water permeable implant to a subject; and
sustainably releasing the drug from the solid water permeable implant for at least about one week following administration of the solid water permeable implant.

8. The method of claim 7, wherein the solid water permeable implant exhibits a reduced one day cumulative drug release compared to a second solid water permeable implant comprising the water permeable polymer and the osmotically active drug formulation that comprises the drug; wherein the second solid water permeable implant has a ratio R of bulk density of the solid water permeable implant to osmotic pressure of the drug formulation wherein R is less than 0.244 grams/milliliter-atm.

9. The method of claim 7, wherein the drug is sustainably released from the solid water permeable implant for at least about two weeks following administration of the solid water permeable implant.

10. The method of claim 6, wherein the water permeable polymer comprises a poly(lactide), poly(glycolide), poly(lactide-co-glycolide), poly(lactic acid), poly(glycolic acid), poly(lactic acid-co-glycolic acid), polyanhydride, polyorthoester, polyetherester, polyethylene glycol, polycaprolactone, polyesteramide, polyphosphazine, polycarbonate, polyamide, or a copolymer or blend thereof.

11. The method of claim 6, wherein the drug comprises leuprolide acetate.
